(19) United States
(12) Patent Application Publication
(10) Pub. No.: US 2008/0097445 A1
(43) Pub. Date: Apr. 24, 2008

(54) BONE FIXATION SYSTEM

(76) Inventor: Robert B. Weinstein, Atlanta, GA (US)

Correspondence Address: BROOKS KUSHMAN P.C., 1000 TOWN CENTER, TWENTY-SECOND FLOOR, SOUTHFIELD, MI, 48075, US

(21) Appl. No.: 11/876,027

(22) Filed: Oct. 22, 2007

Related U.S. Application Data
(60) Provisional application No. 60/853,619, filed on Oct. 23, 2006.

(51) Int. Cl. A61B 17/58; A61B 17/00

(52) U.S. Cl. 606/69; 606/151; 606/61

(57) ABSTRACT

A bone fixation system includes a plurality of bone plate elements, each of which includes a plurality of connecting members. At least one of the bone plate elements includes at least one of a first type of the connecting members, and at least one other of the bone plate elements includes at least one of a second type of the connecting members. Each of the at least one first type of connecting members is configured to cooperate with a respective one of the second type of connecting members to connect at least two of the bone plate elements together, thereby forming a bone plate arrangement. At least some of the bone plate elements have an aperture therethrough for receiving a fastener to affix the bone plate arrangement to a bone.

### CROSS-REFERENCE TO RELATED APPLICATIONS

This application claims the benefit of U.S. provisional patent application Ser. No. 60/853,619 filed on 23 Oct. 2006, which is hereby incorporated herein by reference.

### BACKGROUND OF THE INVENTION

1. Field of the Invention

The present invention relates to a bone fixation system.

2. Background Art

Fixation of a bone with a plate is often performed using an internal device mounted directly to the bone adjacent a fracture, osteotomy, or arthrodesis. The plate is affixed to the bone with fasteners, usually bone screws, spaced along the length of the plate. Current plating systems generally employ a straight or linear plate of varied lengths. In some instances, specially configured or shaped plates are employed when a linear plate would not be appropriate. One example is a calcaneal perimeter plate, which has a shape similar to the normal contour of a calcaneus bone; however, it is useful only for reconstruction of the lateral wall of this bone.

Despite the usefulness of linear plates in some applications, the surgeon often encounters a situation where a using linear plate would result in suboptimal fixation of the bones. For example, the plate may lie over a region of a bone defect where a corresponding bone screw has no bone to engage beneath the plate, which renders that particular plate hole useless. There are also regions of the skeleton where fractured bones are irregular in shape, and therefore, fixation must be non-linear. Such a situation can be seen in comminuted tarsal bone fractures, and with crush injuries generally. Secondary fixation for augmentation of planar joint fusion may also follow a nonlinear path. One example is the medial column fusion often performed for repair of a flatfoot deformity, where restoration of the arch, which is by definition a curved structure, is a primary goal of surgery and is not amenable to long linear plating.

For the purpose of angled plating, reconstruction plates have been developed. The current methods of reconstruction plating allow for bending of some plates and changing the length of certain plates by cutting off the ends with wire cutters or nippers. Bendable plates are generally very thick and prominent in areas of the body where there is little soft tissue coverage. The plate is required to be thick to preserve the integrity of the plate when it is bent. Thicker plates commonly result in discomfort to the patient, and very often these plates require a secondary operation to remove them. Cutting a plate for a specific need can result in weakness of a plate and exposed roughened metal surfaces that are more prone to fretting corrosion.

Plates generally come in sets for use by the surgeon. Often these sets have slots for the various plate sizes, which can be numerous. Because there are necessarily many plate sizes to accommodate many different surgical applications, a hospital or surgical facility must maintain considerable stock. Specially formed or shaped plates, although sometimes available, are also not generally kept in large inventories due to the expense and relative infrequency of use. Therefore, when there is a need for this type of specialty plating, or if inventory of a particular size plate is exhausted, it may not be available to the surgeon, potentially compromising the operation.

Compression of bone ends using a plate is a desired function in many instances of fusion or fracture repair. Fusion in this manner has been the focus of a variety of systems in use for the axial skeleton, such as in cervical vertebral fusion. Plating in the appendicular skeleton has not enjoyed as much attention. It can be advantageous, however, to achieve fusion in planar joint arthrodesis using axial compression rather than oblique compression. Internal fixation using screws across planar joints can create an element of shear where the vector of force of the screws is oblique to the fracture. As an alternative to internal screws, external fixators are employed as a reliable means of creating direct axial compression across the fusion site; however, their use in the extremities is limited for elective fusion and reconstruction. This is due to their cumbersome nature and relatively high potential for complications.

Therefore, a need exists for a bone fixation system that provides geometric flexibility and different modalities to accommodate different fracture patterns and sizes, and reduces the need for large inventories made up of many different sizes and shapes of bone plates.

### SUMMARY OF THE INVENTION

The present invention provides a bone fixation system that uses a number of bone plate elements to create bone plate arrangements of different shapes and sizes to accommodate different surgical applications. For example, embodiments of the invention include bone plate elements having connecting members attached thereto for connecting the bone plate elements to each other with a desired configuration.

Embodiments of the invention include bone plate elements of various geometric shapes, for example, polygons such as hexagons and rectangles. At least some of the edges of these geometric elements have connecting members attached thereto. One or more of the connecting members of a bone plate element may be oriented at an oblique angle from a connecting member along another edge of the bone plate element. In the case of a rectangular bone plate element, two connecting members may be located along opposite sides of the rectangle, or along adjacent sides, essentially perpendicular to each other. In the case of hexagonal bone plate elements, it may be convenient to have three or more of the sides of the hexagon configured with connecting members, thereby adding flexibility to the shapes that can be made by connecting the bone plate elements to each other to form a bone plate arrangement. Unless otherwise stated, the term “bone plate arrangement” as used herein generally refers to an aggregation of bone plate elements that is intended for fixation to a bone proximate a fracture or other defect.

Embodiments of the invention also include a compression member configured to cooperate with at least one of the bone plate elements to provide a means not only for fixation of a fracture, but also to apply compression to the fracture as it heals. The compression member can be configured with a receiving portion to receive one or more of the bone plate elements therein. The receiving portion can be configured to cooperate with one or more of the connecting members on the bone plate element, and further, can be configured with a high-friction surface to help maintain compression and prevent dislocation of the bone plate element after it is disposed within the receiving portion.

Bone plate elements in accordance with the invention may be configured as different geometric shapes, each having cooperating connecting members. In this way, for example, hexagonal bone plate elements can be connected to rectangular bone plate elements to further increase the number of configurations possible for the bone plate arrangements.

Embodiments of the present invention include bone plate elements having connecting members that are configured as dovetails to cooperate with other connecting members. Specifically, some of the connecting members will be elongate members, for example, with a triangular or circular cross section, while mating connecting members will be a triangular or circular shaped groove configured to receive the elongate connecting member on the other bone plate element.

Embodiments of the invention also provide a bone fixation system that includes a plurality of bone plate elements, each of which includes a plurality of connecting members. At least one of the bone plate elements includes at least one of a first type of the connecting members, and at least one other of the bone plate elements includes at least one of a second type of the connecting members. Each of the at least one first type of connecting members is configured to cooperate with a respective one of the second type of connecting members to connect at least two of the bone plate elements together, thereby forming a bone plate arrangement. At least some of the bone plate elements have an aperture therethrough for receiving a fastener to affix the bone plate arrangement to a bone. It is understood that bone plates having such an aperture may have more than one aperture, thereby providing options with regard to fixation.

Embodiments of the invention also include a bone plate arrangement that includes a plurality of bone plate elements chosen from a set of bone plate elements. Each of the chosen bone plate elements cooperates with at least one other of the bone plate elements to form the bone plate arrangement. Each of the chosen bone plate elements defines a polygon having respective sides and a plurality of connecting members disposed along respective sides. Each of the connecting members on one of the chosen bone plate elements is configured to cooperate with one of the connecting members on another of the chosen bone plate elements, such that each of the chosen bone plate elements is connectable to at least two other of the chosen bone plate elements. Embodiments of the invention also include a bone fixation system that can be used to form such a bone plate arrangement, and can further include a compression element such as described above, for use in surgical applications requiring a compressive force to be imparted to a fracture during healing.

### BRIEF DESCRIPTION OF THE DRAWINGS

FIG. 1A is a perspective view of a bone fixation system including a number of hexagonal bone plate elements in accordance with embodiments of the present invention;

FIG. 1B is a top plan view of one of the bone plate elements shown in FIG. 1A;

FIG. 1C is a cross-sectional view of the bone plate element shown in FIG. 1B, taken through line 1C-1C;

FIG. 2 is a top plan view of two of the hexagonal bone plate elements in accordance with embodiments of the present invention connected together;

FIG. 3A is a perspective view of a rectangular bone plate element in accordance with embodiments of the present invention;

FIG. 3B is a top plan view of the bone plate element shown in FIG. 3A;

FIG. 3C is a cross-sectional view of the bone plate element shown in FIG. 3B, taken through line 3C-3C;

FIG. 4 is a partially fragmentary cross-sectional view of a bone plate arrangement in accordance with embodiments of the present invention made up of two bone plate elements attached to a bone adjacent a fracture in the bone;

FIG. 5A is a top plan view of a number of hexagonal bone plate elements connected to each other to form a linear bone plate arrangement in accordance with embodiments of the present invention;

FIG. 5B is a perspective view of the bone plate arrangement of FIG. 5A shown in a flexed position;

FIG. 6 is a top plan view of a bone plate arrangement in accordance with embodiments of the present invention made up of two hexagonal bone plate elements and one rectangular bone plate element;

FIG. 7 is a top plan view of a non-linear bone plate arrangement in accordance with embodiments of the present invention made up of three hexagonal bone plate elements;

FIG. 8A is a top plan view of the bones of a foot having a non-linear mid foot fracture;

FIG. 8B is a top plan view of a bone plate arrangement in accordance with embodiments of the present invention made up of a number of hexagonal bone plate elements and a number of rectangular bone plate elements, and attached to the bones of a foot;

FIG. 9A is a top plan view of a rectangular bone plate element in accordance with embodiments of the present invention;

FIG. 9B is a front plan view of the bone plate element of FIG. 9A illustrating two cylindrical connecting members;

FIG. 10A is a front plan view of another rectangular bone plate element in accordance with embodiments of the present invention, showing the generally cylindrical grooves forming the connecting members configured to receive the connecting members on the bone plate element shown in FIG. 9B;

FIG. 10B is a top plan view of the rectangular bone plate element shown in FIG. 10A;

FIG. 11A is a top plan view of a hexagonal bone plate element in accordance with embodiments of the present invention, including connecting members in the form of grooves shown as hidden lines;

FIG. 11B a top plan view of another hexagonal bone plate element in accordance with embodiments of the present invention, including connecting members configured to be inserted into the connecting members of the bone plate element shown in FIG. 11A;

FIG. 12 is a perspective view of a bone plate element and a compression member in accordance with embodiments of the present invention, for use in stabilizing and inducing compression in a bone fracture;

FIG. 13A is a partially fragmentary cross-sectional view of the compression member and bone plate element of FIG. 12 being applied to a bone fracture; and

FIG. 13B is a partially fragmentary cross-sectional view of the compression member and bone plate element of FIG. 12 installed in the bone in such a way as to compress the fracture in the bone.

### DETAILED DESCRIPTION OF EMBODIMENT(S) OF THE INVENTION

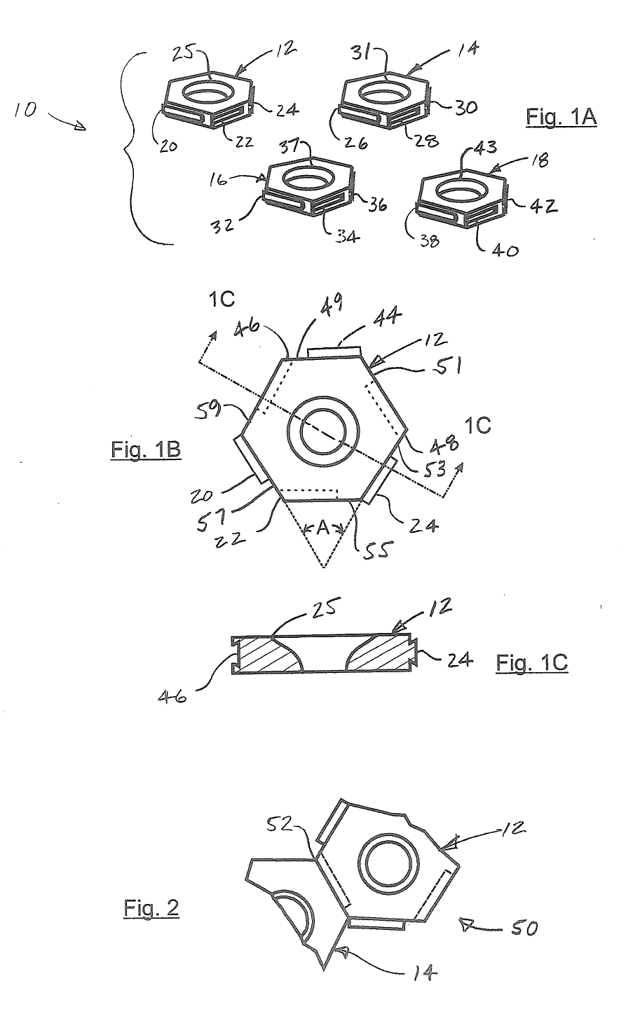

FIG. 1A shows a bone fixation system 10 in accordance with an embodiment of the present invention. The bone fixation system 10 includes a number of bone plate elements 12, 14, 16, 18. Each of the bone plate elements 12, 14, 16, 18 includes a number of connecting members that are configured to cooperate with other connecting members to selectively connect some or all of the bone plate elements 12, 14, 16, 18 to each other. For example, the bone plate element 12 includes connecting members 20, 22, 24. The connecting members 20, 24 are a first type of connecting member, and specifically, a male portion of a dovetail, having a generally triangular cross section. The connecting member 22 is a second type of connecting member, and specifically, a female portion of a dovetail, also having a generally triangular cross section, and configured to receive male dovetail portions, such as the connecting members 20, 24. The bone plate element 12 also includes an aperture 25 disposed therethrough, which can be used to receive a bone screw to affix the bone plate element 12 to a patient's bone, proximate a fracture.

Similarly, the bone plate element 14 includes connecting members 26, 28, 30, and aperture 31. The bone plate element 16 includes connecting members 32, 34, 36, and aperture 37. Finally, the bone plate element 18 includes connecting members 38, 40, 42, and aperture 43. As shown in FIG. 1A, each of the bone plate elements 12, 14, 16, 18 are generally hexagonal in shape, and although only three of the connecting members are shown for each of the bone plate elements 12, 14, 16, 18, it is understood that each has three additional connecting members on their respective sides not visible in FIG. 1A. Having both male and female connecting members on a single bone plate element reduces inventory by allowing any of the bone plate elements to be connected to any other of the bone plate elements.

FIG. 1B is a top plan view of the bone plate element 12 shown in FIG. 1A. In this view, all three of the male dovetail connecting members 20, 24, 44, and all three of the female dovetail connecting members 22, 46, 48 are illustrated. The bone plate element 12 defines six edges 49, 51, 53, 55, 57, 59, which, in the embodiment shown in FIG. 1B, are the sides of the hexagon defined by the bone plate element 12. Because the bone plate element 12 is generally hexagonal, the sides 53, 57 are separated by an angle (A) of approximately 60°. By having the connecting members 20, 24 disposed along respective sides 57, 53, which are at an oblique angle to each other, it is possible to connect a number of bone plate elements together to form a nonlinear bone plate arrangement.

FIG. 1C is a cross-sectional view of the bone plate element 12 taken through line 1C-1C in FIG. 1B. Clearly illustrated in FIG. 1C is the triangular shape of the female dovetail defined by the connecting member 46, and the male dovetail defined by the connecting member 24. Also shown in FIG. 1C is that the aperture 25 includes a countersink to facilitate use of flat or oval head bone screws. Two of the bone plate elements 12, 14 are illustrated in FIG. 2 connected together to form a bone plate arrangement 50 in accordance with the present invention. Depending on manufacturing dimensions and tolerances, an interface 52 between the bone plate elements 12, 14 can be very rigid, or can provide flexibility.

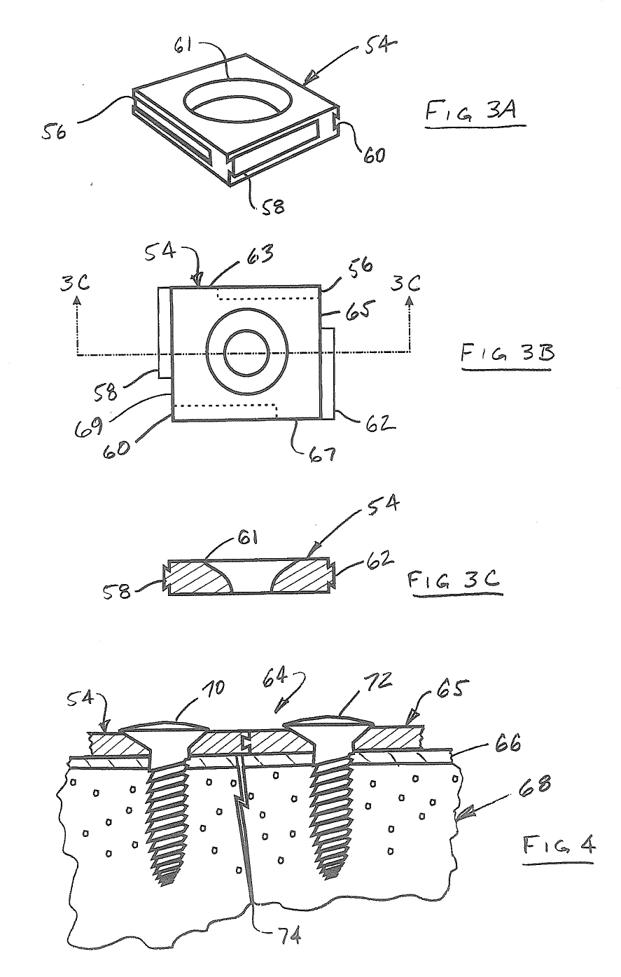

FIG. 3A shows a rectangular bone plate element 54, generally configured as a square in FIG. 3A. The bone plate element 54 includes connecting members 56, 58, 60, and an aperture 61 for receiving a fastener, such as a bone screw. As shown in FIG. 3B, the bone plate element 54 includes a fourth connecting member 62, disposed opposite the connecting member 58. Similar to the bone plate element 12, shown in FIGS. 1 and 2, the bone plate element 54 includes both male dovetail connecting members 58, 62 and female dovetail connectors 56, 60. The connecting members 56, 58, 60, 62 are respectively disposed along sides 63, 69, 67, 65 of the generally rectangular bone plate element 54, shown as edges in FIG. 3B.

As discussed below, embodiments of the present invention contemplate the use of bone plate elements having only one type of connecting member on each element. For example, a bone plate element, such as the bone plate element 54, may be configured with only male dovetail members 58, 62, while another bone plate element, also part of a bone fixation system in accordance with the present invention, may include only female dovetail connecting members, such as the connecting members 56, 60 shown in FIGS. 3A and 3B.

FIG. 3C is a cross-sectional view of the bone plate element 54 taken through line 3C-3C in FIG. 3B. Illustrated in FIG. 3C is the triangular cross section of the connecting members 58, 62, and the countersink formed in aperture 61. FIG. 4 shows a bone plate arrangement 64 made up of two generally rectangular bone plate elements 54, 65. The bone plate arrangement 64 is attached directly to a surface 66 of a patient's bone 68 with bone screws 70, 72. As shown in FIG. 4, the bone plate arrangement 64 helps to stabilize the bone 68 so that a fracture 74 can appropriately heal.

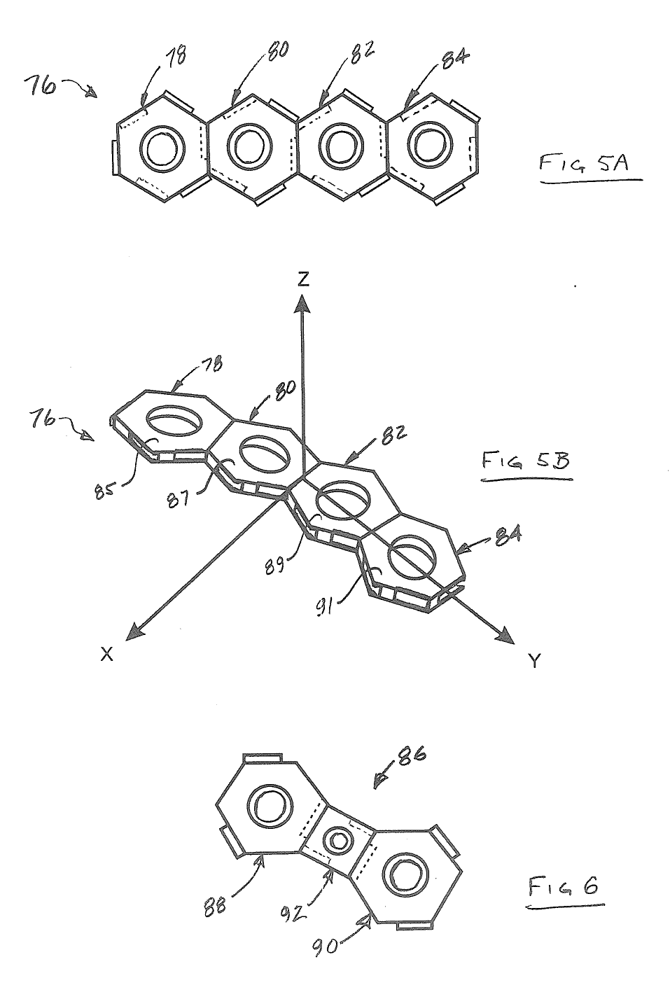

As discussed above, embodiments of the present invention include bone plate arrangements of many different shapes and configurations. For example, FIG. 5A shows a linear bone plate arrangement 76 made up of four generally hexagonal bone plate elements 78, 80, 82, 84. As shown in FIG. 5A, these bone plate elements 78, 80, 82, 84 are configured similarly to the hexagonal bone plate elements 12, 14, 16, 18 shown in FIG. 1A. As discussed above, a bone plate arrangement, such as the bone plate arrangement 76, may have more or less flexibility depending on the relative movement allowed between the bone plate elements, such as the elements 78-84. The amount of flexibility can be controlled by a number of parameters such as the dimensions and tolerances of the various connecting members on the various bone plate elements. Moreover, the sides of the elements, such as the sides 49, 51, 53, 55, 57, 59, of the bone plate element 12 shown in FIG. 1C, may be slightly rounded to afford more flexibility between the elements. For example, FIG. 5B shows the bone plate arrangement 76 having enough flexibility to rotate slightly about the x-axis and the y-axis. Such flexibility can allow a bone plate arrangement, such as the bone plate arrangement 76, to conform to variations in bone surface. Another way this can be accomplished is to vary the surface of the bone plate elements. For example, the surfaces 85, 87, 89, 91 of the bone plate elements 78, 80, 82, 84 can be manufactured with a slight concavity or convex geometry to further accommodate irregular bone surfaces.

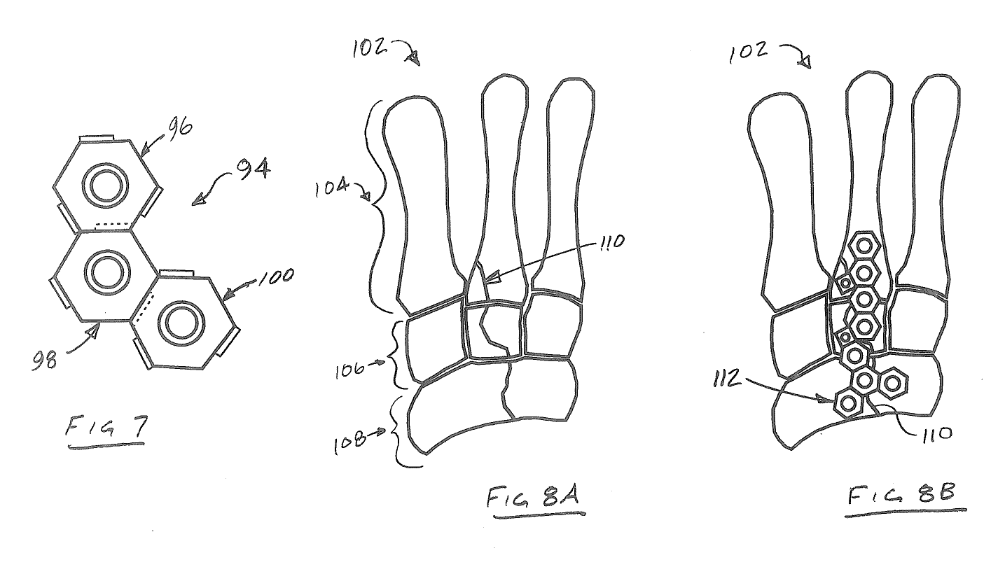

FIG. 6 shows a bone plate arrangement 86 in accordance with an embodiment of the present invention. The bone plate arrangement 86 is made up of three bone plate elements 88, 90, 92, of which elements 88, 90 are generally hexagonal, while element 92 is generally rectangular. This example further illustrates the flexibility of the present invention, which contemplates the use of bone plate arrangements, such as the bone plate arrangement 86, made up of differently shaped bone plate elements having connecting members designed to cooperate with the connecting members of other bone plate elements. In this way, the bone plate elements 88, 90, 92 can be chosen from a large set of bone plate elements, which may for example include the elements 12, 14, 16, 18 shown in FIG. 1A, and a number of generally rectangular elements, such as the rectangular elements 54, 65 shown in FIG. 4, to create bone plate arrangements of different shapes and sizes that are efficacious in the treatment of irregular bone fractures. For example, FIG. 7 shows a bone plate arrangement 94 made up of three bone plate elements 96, 98, 100. Although each of the bone plate elements 96, 98, 100 is generally hexagonal in shape, they are arranged such that the bone plate arrangement 94 is non-linear, and can be used to treat a non-linear fracture.

FIG. 8A shows a part of a skeleton 102 of a human foot, made up of metatarsal bones 104, cuneiform bones 106, and a navicular bone 108. As shown in FIG. 8A, the skeleton 102 is subject to a mid-foot fracture 110. FIG. 8B illustrates the fracture 110 completely stabilized by a bone plate arrangement 112 made up of hexagonal and rectangular bone plate elements as described and illustrated above. Although a preoperative x-ray may provide a surgeon with some indication of the shape of a fracture, use of individual bone plate elements to create a bone plate arrangement, such as the bone plate arrangement 112, allows the surgeon to assemble the bone plate arrangement during surgery, thereby maximizing stabilization while minimizing the use of bone plate material. For example, the use of long linear bone plates to stabilize the fracture 110 shown in FIGS. 8A and 8B would not be able to provide the custom fit stabilization provided by the bone plate arrangement 112, and would necessarily result in the implantation of more plate material, because the linear plates would not efficiently follow the shape of the fracture like the bone plate arrangement 112.

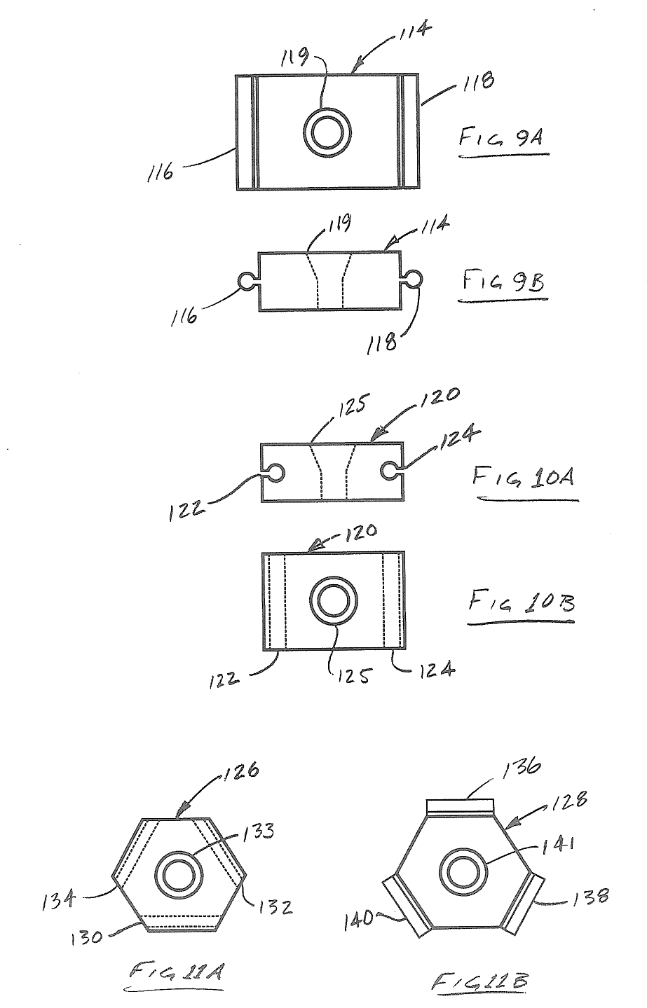

As described and illustrated thus far, the various bone plate elements have each had male and female dovetail portions, and these dovetail portions have each had generally triangular cross sections. Bone fixation systems and bone plate arrangements in accordance with embodiments of the present invention may have any number of different geometric configurations. For example, FIG. 9A shows a top plan view of a bone plate element 114 having connecting members 116, 118, and an aperture 119. The front view of the bone plate element 114, shown in FIG. 9B, clearly illustrates that the connecting members 116, 118 have a generally circular cross section, such that they are generally cylindrical in shape. Moreover, it is shown in FIG. 9A, that each of the connecting members 116, 118 are disposed along an entire side of the bone plate element 114, rather than just a portion of a side, such as described and illustrated above.

FIGS. 10A and 10B illustrate a bone plate element 120 configured to cooperate with the bone plate element 114 to create bone plate arrangements. In particular, the bone plate element 120 includes connecting members 122, 124, which are configured as grooves having a generally circular cross section. As shown in FIG. 10B, the connecting members 122, 124 traverse the entire length of a side of the bone plate element 120. As shown in FIGS. 9 and 10, each of the bone plate elements 114, 120 contains only connecting members of one type—i.e., the bone plate element 114 contains only male connecting members, while the bone plate element 120 contains only female connecting members. Therefore, in bone fixation systems in accordance with the present invention that include bone plate elements having only one type of connecting member, it is contemplated that a set of bone plate elements will include at least two different types of bone plate elements, which may increase inventories, but reduce overall manufacturing costs by eliminating the need to fit both types of connecting members on each bone plate element.

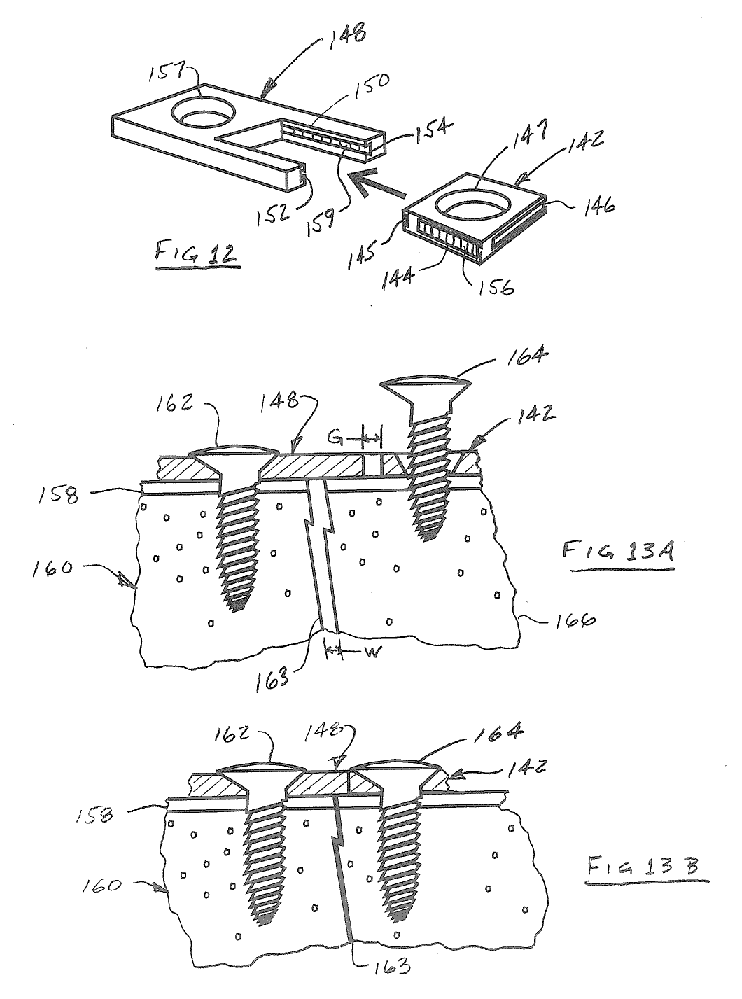

In addition to the generally rectangular bone plate elements, such as the bone plate elements 114, 120 illustrated in FIGS. 9 and 10, a set of bone plate elements may also include other geometric shapes, such as the bone plate elements 126, 128, shown in FIGS. 11A and 11B. The generally hexagonal bone plate element 126 includes three connecting members, 130, 132, 134, each of which is configured with a generally circular cross section, such as the connecting members 122, 124 shown in FIG. 10. The bone plate element 126 also includes an aperture 133 configured with a countersink to accommodate a bone screw. As shown in FIG. 11B, the bone plate element 128 also includes three connecting members, 136, 138, 140, each of which is configured generally the same as the connecting members 116, 118 shown in FIG. 9. The bone plate element 128 also includes an aperture 141 configured with a countersink to accommodate a bone screw.

Each of the bone plate elements 114, 120, 126, 128 can be used to make up a set of bone plate elements from which a number are chosen to create a bone plate arrangement, such as the bone plate arrangement 112 illustrated in FIG. 8B. Although various sizes and shapes of bone plate elements are contemplated by the present invention, bone plate elements effective to perform the functions described herein may have a length of one side, whether rectangular or hexagonal, of somewhere between 5 millimeters (mm) and 20 mm, although other sizes may be used. Bone plate elements of this size may include apertures to receive bone screws ranging from 2 mm to 7 mm. Various thicknesses of material can be used for bone plate elements, although a thickness of 2 mm can be effective when the bone plate elements are made from stainless steel or titanium alloys. Of course, other types of materials effective to perform the intended function can be used.

The various bone plate arrangements described above can be effective to stabilize a bone fracture as it heals. Embodiments of the present invention also contemplate a use of the bone plate elements to apply a compressive force to a fracture to further augment the healing process. FIG. 12 illustrates a generally rectangular bone plate element 142 having connecting members 144, 145, 146. The bone plate element 142 is configured similarly to the bone plate element 54 illustrated in FIG. 3, including an aperture 147 having a countersink configuration. A compression member 148, also part of a bone fixation system in accordance with embodiments of the present invention, includes a receiving portion 150 generally shaped as an open rectangle, configured to receive the bone plate element 142. The receiving portion includes connecting members 152, 154, configured as female dovetail portions with generally triangular cross sections. These are configured to receive the connecting member 144, which is a male dovetail portion, and another connecting member similarly configured on the side opposite the connecting member 144, and therefore not visible in FIG. 12.

As shown in FIG. 12, the connecting member 144 has a high-friction surface 156. Also shown is that the connecting member 154 has a high-friction surface 159. It is understood that the other connecting members will have similar high-friction surfaces that will aid in maintaining a compressive force on a fracture. The high-friction surfaces can be manufactured in any number of ways, including a simple roughening of a machined surface, or with some more elaborate configuration, such as teeth. Moreover, the dimensions of the mating connecting members can be varied to provide different levels of an interference fit.

FIGS. 13A and 13B illustrate the use of the compression member 148 and the bone plate element 142. Initially, the compression member 148 is attached to a surface 158 of a bone 160 with a bone screw 162. Next, the bone plate element 142 is inserted part way into the receiving portion 150 of the compression member 148. The bone plate element 142 is inserted until there remains between the bone plate element 142 and the end of the receiving portion 150 a gap (G) of approximately the same size as a width (W) of a fracture, such as the fracture 163 shown in FIG. 13A.

After the bone plate element 142 is inserted to the appropriate position, a second bone screw 164 is inserted part way into the bone 160. The bone plate element 142 is then driven further into the receiving portion 150 of the compression member 148, thereby closing the gap (G). Because the bone screw 164 is already partly embedded into the bone 160, it carries with it the bone fragment 166, thereby closing the fracture 163. The bone screw 164 is then fully seated into the bone plate element 142, and the high-friction surfaces, such as the surfaces 156, 159, on the connecting members of the bone plate element 142 and the compression member 148 help to maintain the compressive force on the fracture 163. Thus, the configuration illustrated in FIGS. 12 and 13 provide another mechanism by which the present invention provides advantages over known bone fixation systems.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A bone fixation system, comprising:
a plurality of bone plate elements, each of the bone plate elements including a plurality of connecting members,
at least one of the bone plate elements including at least one of a first type of the connecting members, and at least one other of the bone plate elements including at least one of a second type of the connecting members,
each of the at least one first type of connecting members being configured to cooperate with a respective one of the second type of connecting members to connect at least two of the bone plate elements together, thereby forming a bone plate arrangement, and
at least some of the bone plate elements having an aperture therethrough for receiving a fastener to affix the bone plate arrangement to a bone.

2. The bone fixation system of claim 1, wherein a first of the bone plate elements includes a plurality of the first type of connecting members, thereby facilitating connection to at least two other of the bone plate elements each including at least one of the second type of connecting members, thereby forming a bone plate arrangement having at least three of the bone plate elements.

3. The bone fixation system of claim 2, wherein at least two of the first type of connecting members associated with the first bone plate are oriented at an angle to each other to facilitate formation of a non-linear bone plate arrangement.

4. The bone fixation system of claim 1, wherein some of the bone plate elements include only the first type of connecting members and some other of the bone plate elements include only the second type of connecting members.

5. The bone fixation system of claim 1, wherein at least some of the bone plate elements include at least one of the first type of connecting member and at least one of the second type of connecting member.

6. The bone fixation system of claim 1, wherein each of the bone plate elements includes a plurality of edges, and each of the connecting members is disposed along a respective edge of a respective one of the bone plate elements.

7. The bone fixation system of claim 6, wherein each of the first type of connecting members includes a generally cylindrical portion having an axis generally parallel to a respective edge of a respective one of the bone plate elements, and each of the second type of connecting members includes a groove configured to receive the generally cylindrical portion of a respective one of the first type of connecting members.

8. The bone fixation system of claim 1, further comprising a compression member including a receiving portion configured to receive one of the bone plate elements therein, the receiving portion including a high-friction surface for retaining the bone plate element after it is received in the receiving portion.

9. A bone plate arrangement, comprising:
a plurality of bone plate elements chosen from a set of bone plate elements, each of the chosen bone plate elements cooperating with at least one other of the chosen bone plate elements to form the bone plate arrangement,
each of the chosen bone plate elements defining a polygon having respective sides and including a plurality of connecting members disposed along respective sides, each of the connecting members on one of the chosen bone plate elements being configured to cooperate with one of the connecting members on another of the chosen bone plate elements, such that each of the chosen bone plate elements is connectable to at least two other of the chosen bone plate elements, and
at least some of the chosen bone plate elements having an aperture therethrough for receiving a fastener to affix the bone plate arrangement to a bone.

10. The bone plate arrangement of claim 9, wherein at least one of the chosen bone plate elements generally defines a hexagon, and includes at least two of the connecting members disposed along respective sides oriented approximately sixty degrees from each other.

11. The bone plate arrangement of claim 9, wherein at least one of the chosen bone plate elements defines a hexagon and includes at least three of the connecting members disposed along respective sides, each of the three sides being oriented approximately sixty degrees from each of the other two sides.

12. The bone plate arrangement of claim 9, wherein at least one of the chosen bone plate elements generally defines a rectangle, and at least two of the connecting members of a respective generally rectangular bone plate element are disposed along respective edges oriented approximately parallel to each other.

13. The bone plate arrangement of claim 12, wherein at least one of the chosen bone plate elements generally defines a hexagon such that the bone plate arrangement includes at least one hexagonal bone plate element and at least one rectangular bone plate element.

14. The bone plate arrangement of claim 9, wherein at least one of the chosen bone plate elements includes at least one of a first type of the connecting members, and at least one other of the chosen bone plate elements includes at least one of a second type of the connecting members, each of the at least one first type of connecting members being configured to cooperate with a respective one of the second type of connecting members to connect the chosen bone plate elements together.

15. The bone plate arrangement of claim 14, wherein each of the first type of connecting members includes a generally cylindrical portion having an axis generally parallel to a respective side of a respective one of the bone plate elements, and each of the second type of connecting members includes a groove configured to receive the generally cylindrical portion of a respective one of the first type of connecting members.

16. A bone fixation system, comprising:
a plurality of bone plate elements, each of the bone plate elements defining a polygon having respective sides and including a plurality of connecting members disposed along respective sides, each of the connecting members being configured to cooperate with another of the connecting members, such that each of the bone plate elements is connectable to at least two other of the bone plate elements, and
at least some of the bone plate elements having an aperture therethrough for receiving a fastener to affix the respective bone plate element to a bone.

17. The bone fixation system of claim 16, further comprising a compression member including a receiving portion configured to receive one of the bone plate elements therein, the receiving portion including a high-friction surface for retaining the bone plate element after it is received in the receiving portion.

18. The bone fixation system of claim 16, wherein at least one of the bone plate elements includes at least one of a first type of the connecting members, and at least one other of the bone plate elements includes at least one of a second type of the connecting members, each of the at least one first type of connecting members being configured to cooperate with a respective one of the second type of connecting members to connect at least two of the bone plate elements together, thereby forming a bone plate arrangement.

19. The bone fixation system of claim 18, wherein at least two of the first type of connecting members associated with the first bone plate are oriented at an angle to each other to facilitate formation of a non-linear bone plate arrangement.

20. The bone fixation system of claim 16, wherein at least one of the bone plate elements generally defines at least one of hexagon or a rectangle.
